(19) United States
(12) Patent Application Publication
(10) Pub. No.: US 2011/0178450 A1
(43) Pub. Date: Jul. 21, 2011

(54) Manual Spinal Traction Device

(76) Inventor: Paula Mackowiak, Hamburg, NY (US)

(21) Appl. No.: 12/690,914

(22) Filed: Jan. 20, 2010

(51) Int. Cl. A61F 5/00

(52) U.S. Cl. 602/36

(57) ABSTRACT

A manual traction device is disclosed comprising a head-strap configured to wrap around a circumference of a patient's head. A fastener is affixed to the head-strap, configured such that the head-strap may be fastened to itself. The fastener may be a hook-and-loop type fastener. One or more handle-straps are provided, which are slidably attached to the head-strap. In this manner, the position of the handle-strap(s) may be adjusted relative to the length of the head-strap. Using the disclosed manual traction device situated on a patient as more fully described herein, a therapist may apply a pulling force to the handle-strap(s) in a cephalad direction, thereby causing a traction force to be transmitted to the patient's spine.

### FIELD OF THE INVENTION

The present invention relates generally to a device for applying traction to the cervical spinal area, and in particular, to a headgear for manual cervical traction.

### BACKGROUND OF THE INVENTION

Cervical traction is a widely accepted technique used by chiropractors, physical therapists, and other professionals (collectively, “therapists”) for providing pain relief and improving motion in patients. While a patient is lying in a supine position, a trained therapist applies force so as to stretch the cervical spine, opening the spaces between the vertebrae, and elongating the layers of connective tissue throughout the spine. The therapist may use only their hands to perform the technique, or a device for providing traction may be used.

Devices to aid in applying traction force to the cervical region have been designed, but each of the previous attempts suffers certain shortcomings. Some prior art devices utilize halters which generally engage the patient's head in the rear, at the base of the skull, and in the front, under the chin. (See, e.g., U.S. Pat. Nos. 2,984,238, 3,105,489, 3,156,239, and 4,220,147). However, such traction devices may cause discomfort, pain, or even damage to the temporomandibular joint (“TMJ”), the jaw, or the teeth of the patient.

Other devices may engage the patient's head at the base of the skull and wrap around to the patient's forehead. Traction force in these previous devices is applied at the base of the skull. However, such traction devices cannot provide traction force substantially parallel to a central axis of the patient's body. Because the force is applied at the base of the skull, the traction force must be applied at least partially to the front (ventral) side of the patient to prevent slipping.

Still other devices use bail linkages and hinges to create grasping means to apply traction force. (See, e.g., U.S. Pat. No. 5,109,835). Such devices are not designed for manual traction—devices according to this design do not have means for the therapist to manually apply the traction force. Also, because the forces are transmitted through the rigid bail linkages, if a therapist attempted to apply a manual force, the therapist would receive little feedback in the way of “feel.” Therefore, the therapist would have difficulty adjusting the amount and/or direction of the force based on feedback.

Accordingly, there is a need for a device which will allow a wide range of traction force vectors, while providing a secure and comfortable fit for the patient, and a sense of feedback for the therapist. A further need is the ability to easily provide the traction force for an extended period of time.

### BRIEF SUMMARY OF THE INVENTION

The present invention meets the above-described need by providing a manual traction device comprising a head-strap and adjustable handle-strap. The head-strap has a first side, a second side, a fixed end, and a positional end. The head-strap is configured to wrap around a circumference of a patient's head, engaging the base of the skull and the forehead at the supraorbital ridge.

A fastener is affixed to the head-strap, configured such that the head-strap may be fastened to itself. The fastener may be adjustable. The fastener may be a hook-and-loop type fastener.

One or more handle-straps are provided. The handle-strap(s) are slidably attached to the head-strap so that the position of the handle-strap(s) may be adjusted relative to the length of the head-strap. A therapist may apply a pulling force to the handle-strap in a cephalad direction, thereby causing a traction force to be transmitted to the patient's spine. Using two handle-straps, a traction force may be applied to four locations along the length of the head-strap (corresponding to each end of both handle-straps). One having ordinary skill in the art will recognize that a device according to the present invention will allow a therapist to apply a traction force to a patient's cervical spine and/or entire spine.

The handle-straps may further comprise a rigid tube which may be foam covered. In another embodiment, a bar may be provided to engage two handle-straps and allow a therapist to apply force to both handle-straps.

The head-strap may have a high-friction material on the second surface at positions where the head-strap contacts the patient's head. The head-strap may have a padded portion located so as to provide additional comfort to the patient.

### DESCRIPTION OF THE DRAWINGS

For a fuller understanding of the nature and objects of the invention, reference should be made to the following detailed description taken in conjunction with the accompanying drawings, in which:

FIG. 1A is a view of a device according to an embodiment of the present invention, as seen from the first side;

FIG. 1B is a view of the device of FIG. 1A as seen from the second side;

FIG. 2 is a side view of a device according to another embodiment of the present invention, configured on a patient's head;

FIG. 3A is a front view of the device of FIG. 2;

FIG. 3B is a rear view of the device of FIGS. 2 and 3A; and

FIG. 4 is a rear view of a device having a bar according to another embodiment of the present invention, configured on a patient's head.

### DETAILED DESCRIPTION OF THE INVENTION

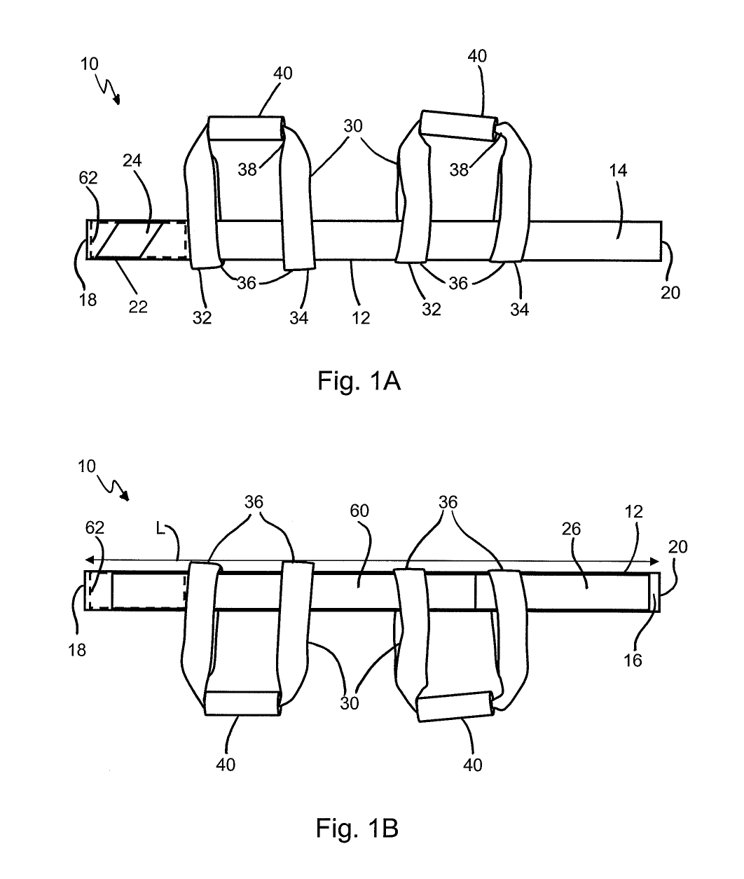

FIGS. 1A and 1B depict a device 10 according to an embodiment of the invention which includes a head-strap 12 having a first side 14, corresponding with an outside of the device 10, and a second side 16, corresponding with an inside (or head-contacting side) of the device 10. The head-strap 12 has a fixed end 18 and a positional end 20. A fastener 22 is affixed to the head-strap 12.

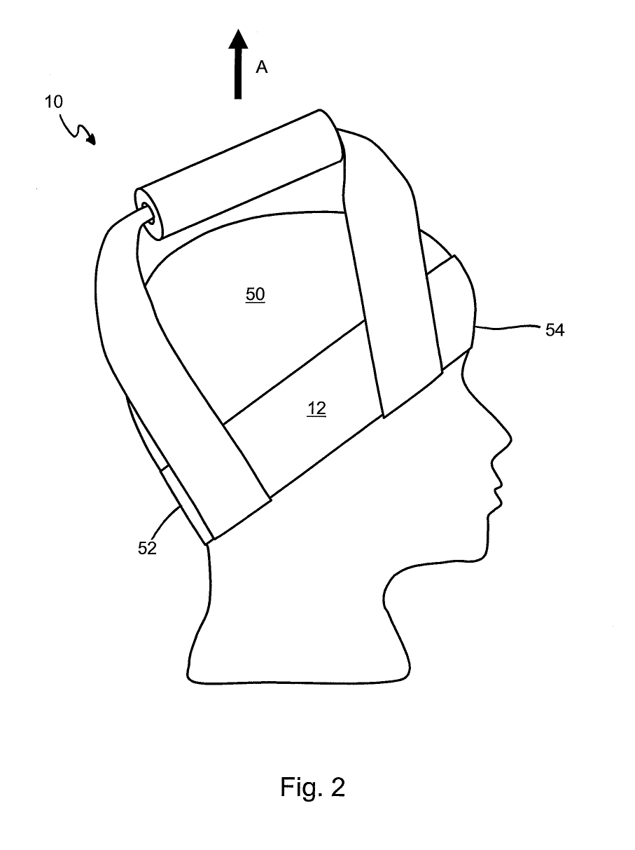

The head-strap 12 is configured to wrap around a circumference of a patient's head. FIG. 2 depicts where, when arranged on a patient's head 50, the head-strap 12 is configured to engage the base of the skull 52 on the caudal side of the head 50, and the forehead at the supraorbital ridge 54 on the rostral side of the head 50. The head-strap 12 may have a length “L” selected such that various patient head sizes may be accommodated The fastener 22 is configured such that the head-strap 12 may be fastened to itself by way of the fastener 22 once the head-strap 12 is wrapped around the patient's head. To accommodate patients having differing head sizes, the fastener 22 is preferably adjustable.

In one embodiment, the fastener 22 is a hook-and-loop type fastener having a hook-type engaging element 24 at the fixed end 18 on the first side 14 of the head-strap 12, and a loop-type engaging element 26 at the positional end 20 on the second side 16 of the head-strap 12. In this way, the head-strap 12 may be placed on the patient by positioning the fixed end 18 on the forehead with the hook-type engaging element 24 facing an outward direction, wrapping the length of the head-strap around the patient's head, and attaching the loop-type engaging element 26 (now facing an inward direction) to the hook-type engaging element 24 such that the head-strap 12 is secured to the patient.

A handle-strap 30 is provided which is slidably attached to the head-strap 12. The handle-strap 30 may be constructed such that a first end 32 and a second end 34 are formed into loop ends 36. In this manner, the head-strap 12 may be inserted through the loop ends 36 such that the handle-strap 30 may be positioned at a desired point along the length of the head-strap 12.

A central portion 38 of the handle-strap 30 may be positioned anteriorly with respect to the patient such that a therapist may apply a pulling force to the central portion 38 of the handle-strap 30 (in the direction indicated by arrow ‘A’ of FIG. 2) and thereby cause a traction force on the patient's spine. It will be recognized by those having skill in the art that using a device 10 of the present invention, force may be applied in directions further to the front, back, and side of the head.

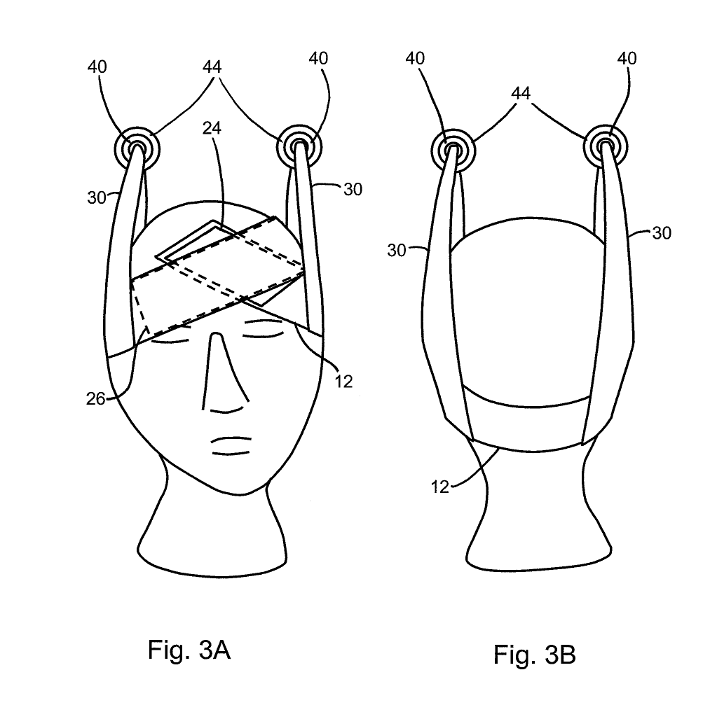

A second handle-strap 30 may be provided (see, e.g., FIGS. 3A and 3B). In this manner, a therapist may use both hands to apply force to four points of the head-strap 12 (corresponding to each end of both handle-straps 30), thereby allowing more complete range in the direction of the traction force.

The handle-straps 30 may further comprise a rigid tube 40 for better gripping by the therapist. The rigid tube may be padded, for example, foam 42 may be disposed on an outside surface of the rigid tube 40.

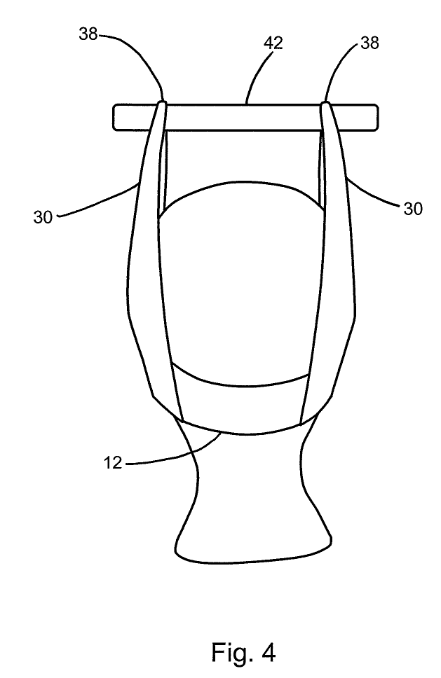

A bar 42 may be provided which may engage the central portions 38 of the handle-straps 30 (see, e.g., FIG. 4). The bar 42 provides further flexibility to the therapist insofar as the manner in which the device 10 is gripped and/or force is applied. For example, by using the bar 42, the therapist may apply force to two handle-straps 30 using only one hand. Also, using the bar 42, the therapist's hand(s) may be oriented generally perpendicular to the position(s) of the therapist's hand(s) when using the handle-straps 30 without the bar 42.

A high-friction material 60 may be disposed on the second surface 16 of the head-strap 12 (see, e.g., FIGS. 1A and 1B). The high-friction material 60, may be located in the head-contacting portions of the head-strap 12. Use of a high-friction material 60 may lessen the likelihood that the position of the device 10 shifts relative to the patient's head when force is applied.

The head-strap 12 may further comprise a padded portion 62 (see, e.g., FIGS. 1A and 1B). The padded portion 62, may be located so as to provide additional comfort to the patient. For example, the padded portion 62 may be located near the fixed end 18 of the head-strap 12.

Although the present invention has been described with respect to one or more particular embodiments, it will be understood that other embodiments of the present invention may be made without departing from the spirit and scope of the present invention. Hence, the present invention is deemed limited only by the appended claims and the reasonable interpretation thereof.

What is claimed is:

1. A spinal traction device for applying traction force to a patient comprising:
a head-strap having a fixed end and a positional end, the head-strap being configured to wrap around a circumference of the patient's head at least at the base of the patient's skull and the supraorbital ridge of the patient's forehead;
a fastener, affixed to the head-strap, for securing the head-strap to itself; and
a handle-strap having a first end and a second end, wherein the first and second ends are slidably attached to the head-strap.

2. The spinal traction device of claim 1, wherein the fastener is a hook-and-loop type fastener.

3. The spinal traction device of claim 2, wherein the fastener includes hook-type engaging elements affixed to the fixed end of the head-strap and loop-type engaging elements affixed to a length of the head-strap at the positional end.

4. The spinal traction device of claim 1, further comprising a second handle-strap having a first end and a second end, wherein the first and second ends are slidably attached to the head-strap.

5. The spinal traction device of claim 4, further comprising a bar configured to engage each of the handle-straps at a central portion of the handle-straps, for allowing a user to apply a traction force to both handle-straps by applying force to the bar.

6. The spinal traction device of claim 1, wherein the handle-strap further comprises a rigid tube around a central portion of the handle-strap.

7. The spinal traction device of claim 6, wherein the rigid tube further comprises foam disposed on an outside surface of the rigid tube.

8. The spinal traction device of claim 1, wherein the head-strap includes a high-friction surface for contacting the patient's head.

9. The spinal traction device of claim 1, wherein the head-strap includes a padded portion.
